SEQUENCE LISTING (11 sequences)

SEQ ID NO: 1 - DNA, 1088 bp, Artificial Sequence
  Recombinant Gene
caatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggatccaaagaggagaaaaagcttatgatgaaaaatattctcgctatccagtctcacgttgtttatggtcatgcgggtaacagtgcggcagagtttccgatgcgccgcctgggcgcgaacgtctggccgctgaacaccgttcaattttctaatcacacccaatacggcaaatggactggctgcgtgatgccgcccagccatttaaccgaaattgtgcaaggcattgccgccattgataaattacacacctgtgatgccgtattaagtggctatctgggatcggcggagcagggtgaacatatcctcggtatcgtccgtcaggtgaaagccgcgaatccgcaggcgaaatatttttgcgatccggtaatgggtcatccggaaaaaggctgtatcgttgcaccgggtgtcgcagagtttcatgtgcggcacggtttgcctgccagcgatatcattgcgccaaatctggttgagctggaaatactctgtgagcatgcggtaaataacgtcgaagaagcggttctggcagcgcgcgaactcattgcgcaagggccacaaattgtgttggttaaacacctggcgcgagctggctacagccgtgaccgttttgaaatgctgctggtcaccgccgatgaagcctggcatatcagccgtccgctggtggattttggtatgcgccagccggtaggtgttggtgatgtgacgagcggtttactgctggtgaaactgcttcagggggcaacgctgcaggaggcgctggaacatgtgaccgctgcagtctacgaaatcatggtgaccaccaaagcaatgcaggaatatgagctgcaagtggtggctgctcaggatcgtattgccaaaccagaacattacttcagcgcaacaaagctctga

SEQ ID NO: 2 - DNA, 2535 bp, Artificial Sequence
  Recombinant Gene
tcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactataggggaattgtgagcggataacaattcccctgtagaaataattttgtttaactttaataaggagatataccatgtctgaattcatggactacaatctcgcgctcgacaaagcgatccagaaactccacgacgagggacgttaccgcacgttcatcgacatcgaacgcgagaagggcgccttccccaaggcgcagtggaaccgccccgatggcggcaagcaggacatcaccgtctggtgcggcaacgactatctgggcatgggccagcacccggtcgttctggccgcgatgcatgaggcgctggaagcggtcggggccggttcgggcggcacccgcaacatctcgggcaccacggcctatcaccgccgtctggaagccgagatcgccgatctgcacggcaaggaagcggcgcttgtcttctcctcggcctatatcgccaatgacgcgacgctctcgacgctgcgcgtgcttttccccggcctgatcatctattccgacagcctgaaccacgcctcgatgatcgaggggatcaagcgcaatgccgggccgaagcggatcttccgtcacaatgacgtcgcccatctgcgcgagctgatcgccgctgatgatccggccgcgccgaagctgatcgccttcgaatcggtctattcgatggatggcgacttcggcccgatcaaggaaatctgcgacatcgccgatgaattcggcgcgctgacctatatcgacgaagtccatgccgtcggcatgtatggcccccgcggcgcgggcgtggccgagcgtgacggtctgatgcaccgcatcgacatcttcaacggcacgctggcgaaagcctatggcgtcttcggcggctacatcgccgcttcggcgaagatggtcgatgccgtgcgctcctatgcgccgggcttcatcttctcgacctcgctgccgccggcgatcgccgctggcgcgcaggcctcgatcgcgtttttgaaaaccgccgaagggcagaagctgcgcgacgcgcaacagatgcacgcgaaggtgctgaaaatgcggctcaaggcgctggggatgccgatcatcgaccatggcagccacatcgttccggtggtcatcggtgaccccgtgcacaccaaggcggtgtcggacatgctcctgtcggattacggcgtttacgtgcagccgatcaacttcccgacggtgccgcgcggcaccgaacggctgcgcttcaccccctcgccggtgcatgacctgaaacagatcgacgggctggttcatgccatggatctgctctgggcgcgctgtgcgctgaatcgcgccgaggcctctgcctga

SEQ ID NO: 3 - DNA, 4688 bp, Artificial Sequence
  Recombinant Gene
tcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactataggggaattgtgagcggataacaattcccctgtagaaataattttgtttaactttaataaggagatataccatgtctgaattcatggactacaatctcgcgctcgacaaagcgatccagaaactccacgacgagggacgttaccgcacgttcatcgacatcgaacgcgagaagggcgccttccccaaggcgcagtggaaccgccccgatggcggcaagcaggacatcaccgtctggtgcggcaacgactatctgggcatgggccagcacccggtcgttctggccgcgatgcatgaggcgctggaagcggtcggggccggttcgggcggcacccgcaacatctcgggcaccacggcctatcaccgccgtctggaagccgagatcgccgatctgcacggcaaggaagcggcgcttgtcttctcctcggcctatatcgccaatgacgcgacgctctcgacgctgcgcgtgcttttccccggcctgatcatctattccgacagcctgaaccacgcctcgatgatcgaggggatcaagcgcaatgccgggccgaagcggatcttccgtcacaatgacgtcgcccatctgcgcgagctgatcgccgctgatgatccggccgcgccgaagctgatcgccttcgaatcggtctattcgatggatggcgacttcggcccgatcaaggaaatctgcgacatcgccgatgaattcggcgcgctgacctatatcgacgaagtccatgccgtcggcatgtatggcccccgcggcgcgggcgtggccgagcgtgacggtctgatgcaccgcatcgacatcttcaacggcacgctggcgaaagcctatggcgtcttcggcggctacatcgccgcttcggcgaagatggtcgatgccgtgcgctcctatgcgccgggcttcatcttctcgacctcgctgccgccggcgatcgccgctggcgcgcaggcctcgatcgcgtttttgaaaaccgccgaagggcagaagctgcgcgacgcgcaacagatgcacgcgaaggtgctgaaaatgcggctcaaggcgctggggatgccgatcatcgaccatggcagccacatcgttccggtggtcatcggtgaccccgtgcacaccaaggcggtgtcggacatgctcctgtcggattacggcgtttacgtgcagccgatcaacttcccgacggtgccgcgcggcaccgaacggctgcgcttcaccccctcgccggtgcatgacctgaaacagatcgacgggctggttcatgccatggatctgctctgggcgcgctgtgcgctgaatcgcgccgaggcctctgcctgactgcagtgcaagcttcatgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgaatattcgtccattgcatgatcgcgtgatcgtcaagcgtaaagaagttgaaactaaatctgctggcggcatcgttctgaccggctctgcagcggctaaatccacccgcggcgaagtgctggctgtcggcaatggccgtatccttgaaaatggcgaagtgaagccgctggatgtgaaagttggcgacatcgttattttcaacgatggctacggtgtgaaatctgagaagatcgacaatgaagaagtgttgatcatgtccgaaagcgacattctggcaattgttgaagcgtaatccgcgcacgacactgaacatacgaatttaaggaataaagataatggcagctaaagacgtaaaattcggtaacgacgctcgtgtgaaaatgctgcgcggcgtaaacgtactggcagatgcagtgaaagttaccctcggtccaaaaggccgtaacgtagttctggataaatctttcggtgcaccgaccatcaccaaagatggtgtttccgttgctcgtgaaatcgaactggaagacaagttcgaaaatatgggtgcgcagatggtgaaagaagttgcctctaaagcaaacgacgctgcaggcgacggtaccaccactgcaaccgtactggctcaggctatcatcactgaaggtctgaaagctgttgctgcgggcatgaacccgatggacctgaaacgtggtatcgacaaagcggttaccgctgcagttgaagaactgaaagcgctgtccgtaccatgctctgactctaaagcgattgctcaggttggtaccatctccgctaactccgacgaaaccgtaggtaaactgatcgctgaagcgatggacaaagtcggtaaagaaggcgttatcaccgttgaagacggtaccggtctgcaggacgaactggacgtggttgaaggtatgcagttcgaccgtggctacctgtctccttacttcatcaacaagccggaaactggcgcagtagaactggaaagcccgttcatcctgctggctgacaagaaaatctccaacatccgcgaaatgctgccggttctggaagctgttgccaaagcaggcaaaccgctgctgatcatcgctgaagatgtagaaggcgaagcgctggcaactctggttgttaacaccatgcgtggcatcgtgaaagtcgctgcggttaaagcaccgggcttcggcgatcgtcgtaaagctatgctgcaggatatcgcaaccctgactggcggtaccgtgatctctgaagagatcggtatggagctggaaaaagcaaccctggaagacctgggtcaggctaaacgtgttgtgatcaacaaagacaccaccactatcatcgatggcgtgggtgaagaagctgcaatccagggccgtgttgctcagatccgtcagcagattgaagaagcaacttctgactacgaccgtgaaaaactgcaggaacgcgtagcgaaactggcaggcggcgttgcagttatcaaagtgggtgctgctaccgaagttgaaatgaaagagaaaaaagcacgcgttgaagatgccctgcacgcgacccgtgctgcggtagaagaaggcgtggttgctggtggtggtgttgcgctgatccgcgtagcgtctaaactggctgacctgcgtggtcagaacgaagaccagaacgtgggtatcaaagttgcactgcgtgcaatggaagctccgctgcgtcagatcgtattgaactgcggcgaagaaccgtctgttgttgctaacaccgttaaaggcggcgacggcaactacggttacaacgcagcaaccgaagaatacggcaacatgatcgacatgggtatcctggatccaaccaaagtaactcgttctgctctgcagtacgcagcttctgtggctggcctgatgatcaccaccgaatgcatggttaccgacctgccgaaaaacgatgcagctgacttaggcgctgctggcggtatgggcggcatgggtggcatgggcggcatgatgtaa

SEQ ID NO: 4 - DNA, 31 bp, Artificial Sequence
  Primer
gcgcttatgagtagtttgttgttgtttaacg

SEQ ID NO: 5 - DNA, 30 bp, Artificial Sequence
  Primer
actagtttatgcttccgccagcggcggcaa

SEQ ID NO: 6 - DNA, 48 bp, Artificial Sequence
  Primer
aggagatataccatgtctgaattcatggactacaatctcgcgctcgac

SEQ ID NO: 7 - DNA, 58 bp, Artificial Sequence
  Primer
ctgttcgacttaagcattatgcggccgtcgactcaggcagaggcctcggcgcgattca

SEQ ID NO: 8 - DNA, 58 bp, Artificial Sequence
  Primer
tgaatcgcgccgaggcctctgcctgagtcgacggccgcataatgcttaagtcgaacag

SEQ ID NO: 9 - DNA, 48 bp, Artificial Sequence
  Primer
gtcgagcgcgagattgtagtccatgaattcagacatggtatatctcct

SEQ ID NO: 10 - DNA, 33 bp, Artificial Sequence
  Primer
tacatatgaatattcgtccattgcatgatcgcg

SEQ ID NO: 11 - DNA, 33 bp, Artificial Sequence
  Primer
cgcgatcatgcaatggacgaatattcatatgta

(19) United States
(12) Patent Application Publication
(10) Pub. No.: US 2023/0193330 A1
(43) Pub. Date: Jun. 22, 2023

(54) ESCHERICHIA COLI FOR PRODUCING 5-AMINO- LEVULINIC ACID AND METHOD OF PRODUCING 5-AMINOLEVULINIC ACID

(71) Applicant: NATIONAL CHENG KUNG UNIVERSITY, Tainan City (TW)

(72) Inventors: I-SON NG, Tainan City (TW); YING-CHEN YI, Tainan City (TW); CHENGFENG XUE, Tainan City (TW); TZU-HSUAN YU, Tainan City (TW)

(21) Appl. No.: 17/695,856

(22) Filed: Mar. 16, 2022

(30) Foreign Application Priority Data
Dec. 20, 2021 (TW) 110147693

(51) Int. Cl. C12P 13/00; C12N 1/20

(52) U.S. Cl. CPC C12P 13/005; C12N 1/205; C12R 2001/19

(57) ABSTRACT

A Escherichia coli strain for producing 5-aminolevulinic which has double pdxY genes is provided. A method of producing 5-aminolevulinic acid is provided, the method comprises providing the above Escherichia coli; and inoculating the above Escherichia coli into liquid modified M9 medium containing carbon source, IPTG, glycine, succinic acid and pyridoxal to cultivate the above Escherichia coli thereby producing 5-amino-levulinic. By the above Escherichia coli and the method of producing 5-aminolevulinic acid, the strain with high growth rate and 5-aminolevulinic productivity and the mothed of quickly producing 5-aminolevulinic acid with high 5-aminolevulinic productivity are provided.

### CROSS-REFERENCE TO RELATED APPLICATION

This non-provisional application claims priority under 35 U.S.C. § 119(a) on Patent Application No(s). 110147693 filed in Taiwan, R.O.C. on Dec. 20, 2021, the entire contents of which are hereby incorporated by reference.

### REFERENCE TO A SEQUENCE LIST

This application refers to a “Sequence list” listed below, which is provided as an electronic document, created on Mar. 10, 2022, entitled “Sequence.txt” and being 12,479 bytes in size, the “Sequence list” is incorporated herein by reference in its entirety.

### BACKGROUND OF THE INVENTION

### 1. Field of the Invention

The present invention relates to an Escherichia coli for producing 5-aminolevulinic acid and a method for producing 5-aminolevulinic acid using the same, and in particular to an Escherichia coli made by a gene transfer method.

### 2. Description of the Related Art

5-aminolevulinic acid (hereinafter referred to as ALA) is an essential metabolic intermediate in organisms. ALA has a wide range of uses in the agricultural field. For example, ALA can be used as a herbicide, an insecticide or a promoting factor for crop growth. In addition, ALA is also used for medical diagnostic purposes. For example, ALA can be used as a marker for cancer cells. The current production method of ALA mainly utilizes Corynebacterium glutamicum to produce ALA.

### BRIEF SUMMARY OF THE INVENTION

However, the growth rate of Corynebacterium glutamicum is relatively slow, and Corynebacterium glutamicum needs a longer culture time to reach a higher bacterial population, thereby increasing its ALA production, which means that the slow growth rate of Corynebacterium glutamicum affects the production efficiency of ALA. Therefore, how to develop a strain that grows fast and has high ALA yield is still an issue to be solved.

An object of the present invention is to solve the above issue and provide an Escherichia coli having double pdxY gene and capable of producing 5-aminolevulinic acid.

As to the Escherichia coli described above, the Escherichia coli further comprises RchemA gene.

As to the Escherichia coli described above, the Escherichia coli further comprises pRARE plasmid.

In order to achieve the above and other objects, the present invention provides an Escherichia coli having the sequence of SEQ ID NO. 1 and capable of producing 5-aminolevulinic acid.

As to the Escherichia coli described above, the Escherichia coli further has the sequence of SEQ ID NO. 2.

As to the Escherichia coli described above, the Escherichia coli further comprises pRARE plasmid.

In order to achieve the above and other objects, the present invention provides a method for producing 5-aminolevulinic acid, comprising: (a) providing the above Escherichia coli; and (b) inoculating the Escherichia coli in a medium containing a carbon source, isopropyl-β-D-thiogalactoside, glycine, succinic acid and pyridoxal for culture to produce 5-aminolevulinic acid.

As to the method described above, the carbon source is a mixed carbon source composed of glucose and glycerol.

As to the method described above, the medium contains iron ions.

Through the Escherichia coli that produces 5-aminolevulinic acid and the method for producing 5-aminolevulinic acid as described above, a strain that grows fast and has high ALA yield and a method capable of producing ALA rapidly with high yield are provided.

### BRIEF DESCRIPTION OF THE DRAWINGS

FIG. 1 shows the CRIM plasmid map used in Example 1 of the present invention.

FIG. 2 shows the SITAG plasmid map used in Example 1 of the present invention.

FIG. 3 shows the plasmid copy number in the ALA production efficiency test of Escherichia coli PIECE according to Example 1 of the present invention.

FIG. 4 shows the biomass in the ALA production efficiency test of Escherichia coli PIECE according to Example 1 of the present invention.

FIG. 5 shows the ALA yield of Escherichia coli PIECE in the ALA production efficiency test of Escherichia coli PIECE according to Example 1 of the present invention.

FIG. 6 shows the pSIT plasmid map used in Example 2 of the present invention.

FIG. 7 shows the comparison results of growth rate of Escherichia coli RcGI according to Example 2 of the present invention and other transgenic Escherichia coli.

FIG. 8 shows the comparison results of ALA yield of Escherichia coli RcGI according to Example 2 of the present invention and other transgenic Escherichia coli.

FIG. 9 shows the comparison results of growth rate of Escherichia coli RcGI according to Example 2 of the present invention with iron ions added at different time points.

FIG. 10 shows the comparison results of ALA yield of Escherichia coli RcGI according to Example 2 of the present invention with iron ions added at different time points.

FIG. 11 shows the mass production efficiency test results of Escherichia coli RcGI in a fermentation tank according to Example 2 of the present invention.

FIG. 12 shows the pCT-PY plasmid map used in Example 3 of the present invention.

FIG. 13 shows the mass production efficiency test results of Escherichia coli A-RcYI in a fermentation tank according to Example 3 of the present invention.

### DETAILED DESCRIPTION OF THE INVENTION

To fully understand the object, features and effects of the present invention, the following examples are provided in conjunction with the accompanying drawings to give a detailed description of the present invention as follows:

Escherichia coli PIECE of Example 1

Example 1 provides an Escherichia coli (E. coli) PIECE. The PIECE strain contains an extra inserted pdxY gene of E. coli, that is, the PIECE strain contains double pdxY genes (its own pdxY gene and an additional pdxY gene). The pdxY gene expresses PdxY protein (namely pyridoxal kinase). PdxY protein can assist the synthesis of pyridoxal phosphate (hereinafter referred to as PLP), and PLP is a cofactor of ALA synthetase (hereinafter referred to as ALAS). In other words, the increase in the expression of pdxY gene contributes to the increase of ALA yield of E. coli. Compared with Corynebacterium glutamicum, E. coli having relatively fast growth rate can grow enough amount of bacteria in reduced time, thereby producing more ALA. The preparation method of the PIECE strain of Example 1 is as follows.

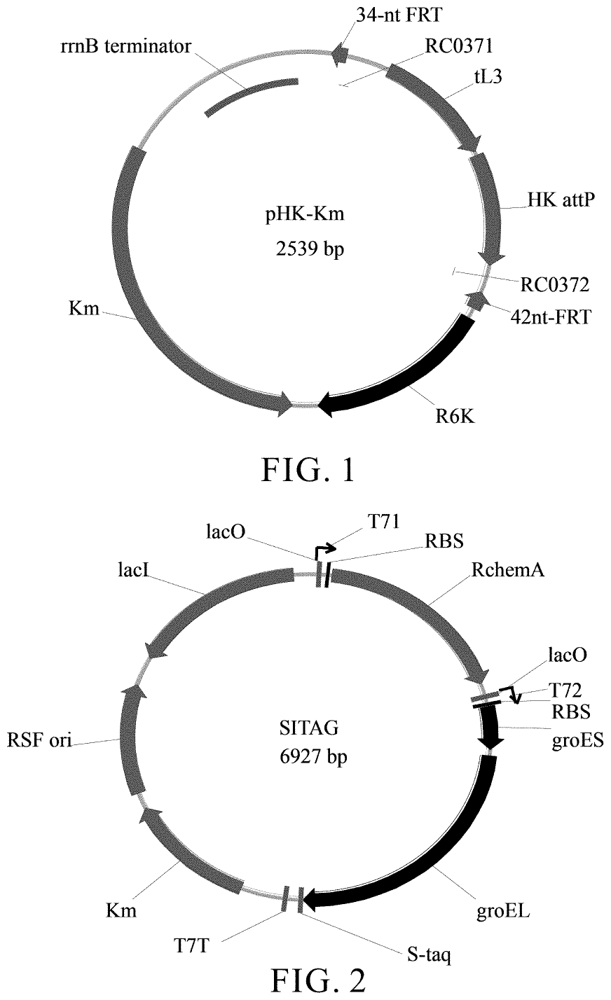

First, the conditional-replication, integration, and modular (CRIM) plasmids and E. coli strain BL21 (DE3) were prepared. As shown in FIG. 1, the CRIM plasmid used in Example 1 was pHK-Km. The CRIM plasmid carried the promoter Lad and pdxY genes to form the pHK-PlacI-pdxY-Km plasmid. The pHK-PlacI-pdxY-Km plasmid of Example 1 carried the kanamycin gene. The CRIM plasmid was transferred into E. coli BL21 (DE3) by transformation.

The preparation of the pdxY gene is amplified by PCR. The primer sequence used for pdxY gene amplification is as follows: the forward primer sequence is 5′-GCGCTTATGAGTAGTTTGTTGTTGTTTAACG-3′; and the reverse primer sequence is 5′-ACTAGTTTATGCTTCCGCCAGCGGCGGCAA-3′.

The above transformation process is that first mixing E. coli BL21 (DE3) and CRIM plasmids, then heat-shocking the mixture of E. coli BL21 (DE3) and CRIM plasmids at 42° C. for 90 seconds, then adding LB solution to the aforementioned mixture to recover for 1 hour, and finally spreading the LB bacterial fluid containing the transformed E. coli BL21 (DE3) on the LB agar medium.

The CRIM plasmid transferred into E. coli BL21 (DE3) can integrate the pdxY gene and the kanamycin gene into the HK022 site of E. coli BL21 (DE3). In this way, the PIECE strain of Example 1 can be produced. The PIECE strain of Example 1 was obtained by culturing the PIECE strain on LB agar medium containing kanamycin and further selection.

In order to avoid that the PIECE strain containing the kanamycin gene was not easy to be eliminated due to drug resistance after entering the environment, it was necessary to further eliminate the kanamycin gene in the PIECE strain. The elimination method was based on the aforementioned transformation conditions, in which the pAH69 plasmid was transferred into the PIECE strain according to the method provided by previous literatures (ANDREAS HALDIMANN and BARRY L. WANNER. Conditional-Replication, Integration, Excision, And Retrieval Plasmid-Host Systems For Gene Structure—Function Studies Of Bacteria. J Bacteriol. 2001 November; 183(21):6384-93.) to remove the kanamycin gene from the PIECE strain using pAH69 plasmids.

Finally, according to the heating method provided in previous literatures (Francois St-Pierre et al. One-Step Cloning and Chromosomal Integration of DNA. ACS Synth Biol. 2013 Sep. 20; 2(9):537-41), all the plasmids remaining in the PIECE strain were removed to obtain the PIECE strain capable of expressing the PdxY protein stably. The DNA of the PIECE strain had the sequence of SEQ ID NO. 1 as shown in the sequence listing. The PIECE strain of Example 1 expresses the pdxY gene through directly embedding the pdxY gene into DNA of the PIECE strain itself instead of the plasmids entering into the PIECE strain. Therefore, the PIECE strain of Example 1 can stably express the pdxY gene.

The above Example 1 discloses that the pdxY gene is transferred into the PIECE strain through CRIM plasmids containing the promoters Lad and RBS to express the pdxY gene. However, in other embodiments, other known vectors, other promoters or other ways can also be used to make the E. coli strain express the pdxY gene, and is not limited to Example 1. In addition, the E. coli strain selected in Example 1 is BL21 (DE3), but in other embodiments, other E. coli strains can also be selected to express the pdxY gene.

ALA Production Efficiency Test of PIECE Strain

First, the PIECE strain was prepared according to the preparation method of the PIECE strain described above; and meanwhile, the original E. coli strain BL21(DE3) without inserting the pdxY gene was prepared. Then, according to the above-mentioned CRIM plasmid transformation method, SITAG plasmids were respectively transferred into PIECE strain and E. coli strain BL21 (DE3), in which SITAG plasmids carried gene clusters (T7-RchemA-T7-GroES-GroEL), RchemA was a gene expressing ALA synthase in Rhodobacter capsulatus, and GroES and GroEL were chaperone proteins that can improve the stability of strain growth. The preparation method of SITAG plasmids is as follows. First, through extended overlap extension PCR, the RchemA gene was inserted into the pSIT plasmid with primers IF, IR, VF, and VR to make pSIT-Rc plasmids. Then, the GroES gene and GroEL gene obtained from E. coli were inserted into the pSIT plasmids with the primers NdeI-GF and XhoI-GR in the aforementioned manner to prepare SITAG plasmids. The sequence of the above primer is shown in Table 1 below. The SITAG plasmid is a plasmid that allows the strain to produce ALA, that is, in this test, the difference in ALA production efficiency between the PIECE strain inserted with the pdxY gene and the general E. coli are compared.

TABLE 1
The primers used to prepare SITAG plasmids

| Primer | Sequence (5′-3′) |
|---|---|
| IF | AGGAGATATACCATGTCTGAATTCATGGACTACAA |
| | TCTCGCGCTCGAC |
| IR | CTGTTCGACTTAAGCATTATGCGGCCGTCGACTCA |
| | GGCAGAGGCCTCGGCGCGATTCA |
| VF | TGAATCGCGCCGAGGCCTCTGCCTGAGTCGACGGC |
| | CGCATAATGCTTAAGTCGAACAG |
| VR | GTCGAGCGCGAGATTGTAGTCCATGAATTCAGAC |
| | ATGGTATATCTCCT |
| NdeI-GF | TACATATGAATATTCGTCCATTGCATGATCGCG |
| XhoI-GR | CGCGATCATGCAATGGACGAATATTCATATGTA |

Afterwards, the PIECE strain carrying SITAG plasmids (hereinafter referred to as SITAG/PIECE strain) and the BL21(DE3) strain carrying SITAG plasmids (hereinafter referred to as SITAG/BL21(DE3) strain) were respectively inoculated into 2 mL of LB culture medium under 37° C. for 12 hours to serve as the pre-culture medium. The pre-culture media of SITAG/PIECE strain and SITAG/BL21(DE3) strain were taken and inoculated at a ratio of 2% in 30 mL of MM9 culture medium (containing 20 g/L glucose) for culture. The above MM9 culture medium was placed in a 250 mL shake flask, in which the MM9 culture medium containing the SITAG/PIECE strain was used as the experimental group, and the MM9 medium containing the SITAG/BL21(DE3) strain was used as the control group. Both the experimental group and the control group were cultured in a shaking flask with a rotation speed of 175 rpm under 37° C. When the concentration of the bacterial fluids in the experimental group and the control group reached an OD₆₀₀ value of approximately 0.6, 0.1 mM isopropyl-β-D-thiogalactoside (IPTG) was added to the bacterial fluid of each group as a gene expression inducing agent. Also, 4 g/L glycine, 1 g/L succinic acid and 40 μM pyridoxal were added, in which glycine and succinic acid were used as the substrate required for the synthesis of ALA, and pyridoxal served as the precursor of PLP. By using pyridoxal to replace PLP, the strain itself synthesized pyridoxal into PLP, which can reduce the cost of the process. After adding IPTG, glycine, succinic acid and pyridoxal to the experimental group and the control group, the experimental group and the control group were cultivated in a shake flask with a rotation speed of 200 rpm at 30° C. for 24 hours. During the 24 hours of culture, the plasmid copy number, biomass and ALA yield of the experimental and the control groups were measured respectively at different time points. The above process was repeated three times.

The method for measuring plasmid copy number of the experimental group and the control group is as follows. First, after the experimental group and the control group were cultured for 24 hours, the experimental group and the control group were centrifuged at 12,000 rpm for 3 minutes to obtain the bacterial precipitates. The bacterial precipitates of the experimental group and the control group were washed with deionized water twice, and then evenly dispersed in deionized water and placed in high temperature treatment environment at 95° C. for 10 minutes. After the high temperature treatment, the bacterial precipitates of the experimental group and the control group were centrifuged again at 12,000 rpm for 3 minutes, and then the supernatant after centrifugation was taken out. Finally, the EvaGreen reagent (Applied Biosystems, USA) used in the real-time PCR system was used for quantitative PCR (qPCR) analysis. The target gene used in the evaluation of plasmid copy number can be selected from the genes used in previous literatures (Yi YC, Ng IS. Establishment of toolkit and T7RNA polymerase/promoter system in Shewanella oneidensis MR-1. J Taiwan Inst Chem Eng 2020; 109:8-14).

The biomass measurement method for the experimental and the control groups is as follows. At the 2^{nd}, 4^{th}, 8^{th}, 12^{th} and 24^{th} hour of culture of the experimental group and the control group incubated with IPTG, glycine, succinic acid and pyridoxal as described above, 200 μL of bacterial fluid samples were taken out from the experimental group and the control group. At each sampling time point, the OD₆₀₀ values of the bacterial fluids from the experimental group and the control group were determined first, and then the bacterial fluid samples of the experimental group and the control group were centrifuged at a rotation speed of 6000 xg for 10 minutes. After centrifugation, the bacterial precipitates of the experimental group and the control group were taken out and washed twice with deionized water, and then heated and dried by an infrared humidity analyzer (FD-660, Kett Electric Laboratory, Japan) at 110° C. for 20 minutes until the water in the bacterial precipitates of the experimental group and the control group was completely removed to obtain the exact dry weight of the bacterial precipitates, thereby obtaining the unit biomass (g/L) of the experimental group and the control group.

The ALA yield measurement method of the above experimental group and the control group was performed with reference to the experimental procedures and experimental conditions in previous literatures (Ying-Chen Yi, Chengfeng Xue and I-Son Ng, Low-Carbon-Footprint Production of High-End 5-Aminolevulinic Acid via Integrative Strain Engineering and RuBisCo-Equipped Escherichia coli. ACS Sustainable Chemistry & Engineering 2021 9 (46), 15623-15633) and the product operation manual of the Bio-Rad Protein Assay Kit. The ALA yield measurement procedures are summarized as follows.

First, 200 μL of the reaction solution was prepared. The reaction solution was composed of 50 mM Tris HCl (pH 7.5), 50 mM NaCl, 0.1 mM PLP, and 1 mg/mL free enzyme (the crude protein obtained after destruction of cell membrane of bacterium by high-pressure cell disruption), 0.29 mM succinyl coenzyme A and 2 mM glycine.

Next, at the 8^{th}, 12^{th} and 24^{th} hour of culture of the experimental group and the control group incubated with IPTG, glycine, succinic acid and pyridoxal as described above, 2 mL of bacterial fluid sample was taken out from the experimental group and the control group at each sampling time point, followed by addition of 200 μL of 1 M sodium acetate (pH 4.6) and 40 μL of acetylacetone to the reaction mixture of each group to terminate the ALA synthesis reaction performed by ALAS. Afterwards, the reaction mixtures of each group that had terminated the synthesis reaction were heated at 100° C. for 10 minutes, and then further make the ALA produced by the aforementioned synthesis reaction fully react with acetylacetone. After the heating was completed, the temperature was lowered to room temperature, and then an equal amount of Ehrlich reagent was added to the reaction mixture of each group, mixed uniformly and reacted for 10 minutes to quantify the ALA synthesized by each group at each time point. The preparation of Ehrlich reagent referred to previous literatures (Shemin D, Russell CS. d-aminolevulinic acid its role in the biosynthesis of porphyrins and purines. J Am Chem Soc 1953; 75(19):4873-4.) After the reaction of the reaction mixture of each group with Ehrlich reagent was completed, the ALA yield of each group was determined using a micro-disc analyzer with a wavelength of 553 nm.

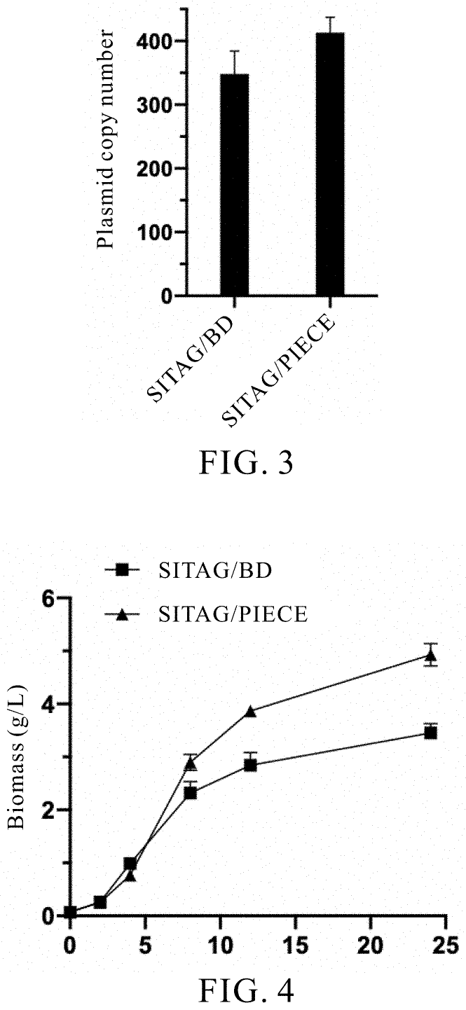

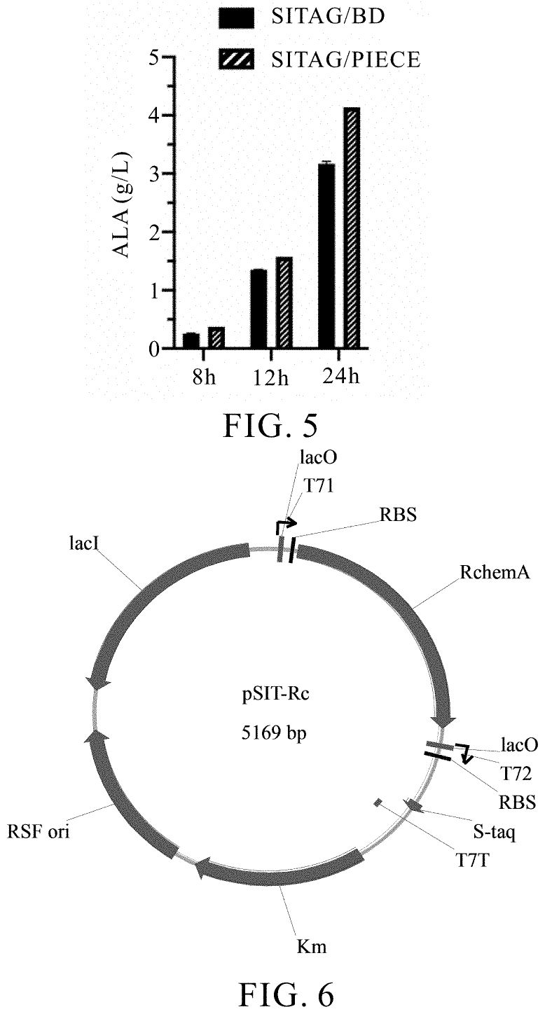

The test results of plasmid copy number, biomass and ALA production efficiency of the above-mentioned PIECE strains are shown in FIGS. 3-5. The SITAG/PIECE strain has higher biomass and ALA yield, compared with the SITAG/BL21(DE3) strain containing only a single set of pdxY gene.

E. coli RcGI of Example 2

Example 2 provides an E. coli RcGI. The RcGI strain contains an additional inserted hemA gene and the chaperonin GroES gene and GroEL gene. As mentioned above, the RchemA gene is a gene expressing ALA synthetase, and the chaperone proteins GroES and GroEL can improve the growth stability of the strain. In Example 2, the RchemA gene, GroES gene, and GroEL gene were transferred into E. coli for further testing the effect in improving E. coli production efficiency of ALA. The preparation method of the RcGI strain in Example 2 is as follows.

First, the CRIM plasmid as shown in FIG. 1 and the E. coli strain BL21 (DE3) were prepared. The CRIM plasmid of Example 2 was further inserted with the gene cluster (T7-RchemA-T7-GroES-GroEL) integrated by the RchemA gene, the GroES gene and the GroEL gene.

Next, according to the transformation method of Example 1, the aforementioned CRIM plasmids carrying the gene cluster T7-RchemA-T7-GroES-GroEL were transferred into E. coli BL21 (DE3) to make RcGI strains. At this time, the gene cluster T7-RchemA-T7-GroES-GroEL was directly integrated into the DNA of E. coli, and the above gene cluster was not expressed through the plasmids that coexist in E. coli and were not integrated into the DNA of E. coli. Furthermore, according to the method of Example 1, the pAH69 plasmid was transferred into the RcGI strain to remove the kanamycin gene in the RcGI strain.

The preparation of the above-mentioned RchemA gene was amplified by PCR reaction. The primer sequences used for RchemA gene amplification were as follows: the forward primer sequence was 5′-TCGACGAAGTCCATGCTGTCGG-3′; and the reverse primer sequence was 5′-CGACGTAGAGAAGATGAATCCAG-3′.

Finally, by removing all the plasmids remaining in the RcGI strain through the heating method as described in Example 1, an RcGI strain capable of stably expressing ALA synthase and the chaperone proteins GroES and GroEL was obtained. At this time, the DNA of the RcGI strain had the sequence of SEQ ID NO. 3 as shown in the sequence listing.

The above-mentioned Example 2 revealed that the RchemA gene, GroES, and GroEL genes were transferred into the RcGI strain through the CRIM plasmid as shown in FIG. 1 to make the RcGI strain express the RchemA gene, GroES gene and GroEL gene, but in other embodiments, other known vectors, other promoters or other methods can also be used to make the E. coli strain express the RchemA gene, GroES gene and GroEL gene, and it was not limited to Example 2. In addition, the E. coli strain selected in Example 2 was BL21 (DE3), but in other embodiments, other E. coli strains could also be used to express the RchemA gene, GroES gene and GroEL gene.

ALA Production Efficiency Test of RcGI Strain

First, the RcGI strain was prepared according to the above-mentioned preparation method of the RcGI strain. Meanwhile, according to the aforementioned transformation method for preparing RcGI strain, the pSIT plasmid as shown in FIG. 6 carried RchemA gene, GroES gene, and GroEL gene into the original E. coli strain BL21 (DE3), thereby making RcG strain. At this time, the RchemA gene, GroES gene, and GroEL gene were expressed through plasmids coexisting in E. coli but not integrated into the DNA of E. coli; and, according to the aforementioned transformation method for preparing RcGI strains, the pSIT plasmid carried the RchemA gene into the original E. coli strain BL21 (DE3), thereby making the Rc strain.

Next, the RcGI strain, RcG strain and Rc strain were respectively inoculated into 2 mL of LB culture medium and cultured under 37° C. for 12 hours to serve as the pre-culture medium. The pre-culture medium of RcGI strain, RcG strain and Rc strain were inoculated respectively into 30 mL of MM9 culture medium (containing 20 g/L glucose and 10 g/L glycerol as the carbon source, in which the weight ratio of glucose to glycerol is 2:1) in a 250 mL Erlenmeyer flask at the inoculation ratio of 2%. In this test, in order to achieve the benefits of inhibiting the activity of ALA dehydratase, increasing the expression level of ALAS and reducing costs, a mixed carbon source composed of glucose and glycerol was used. Glucose was also used as an inhibitor of ALA dehydratase, and glycerol could serve as a second carbon source to increase the expression level of the protein. However, in other embodiments, a single carbon source or other types of carbon sources could still be selected depending on the needs and not limited thereto. After the RcGI strain, RcG strain and Rc strain were inoculated into the MM9 culture media, the above-mentioned RcGI strain, RcG strain and Rc strain were incubated under 37° C. with shaking at 200 rpm. When the concentrations of the bacterial fluids of the above-mentioned RcGI strain, RcG strain and Rc strain were determined by spectrophotometer to reach an OD₆₀₀ value of approximately 0.6, 0.1 mM IPTG, 4 g/L glycine, 1 g/L succinic acid and 50 μM pyridoxal phosphate (PLP) were added to the bacterial fluids of the above-mentioned RcGI strain, RcG strain and Rc strain. After adding IPTG, glycine, succinic acid and pyridoxal to the bacterial fluids of the above-mentioned RcGI strain, RcG strain and Rc strain, the culture was continued for 30 hours at 300-400 rpm in an environment of 30° C. At the 4^{th}, 8^{th}, 12^{th}, 24^{th} and 30^{th} hour time points of culture, the bacterial fluids of the RcGI strain, RcG strain and Rc strain were taken out to determine the OD₆₀₀ values of the RcGI strain, RcG strain and Rc strain by the spectrophotometer, and according to the ALA production efficiency test method of Example 1, the ALA yield of the RcGI strain, RcG strain and Rc strain were determined respectively at the 12th, 24th and 30th hour after culture.

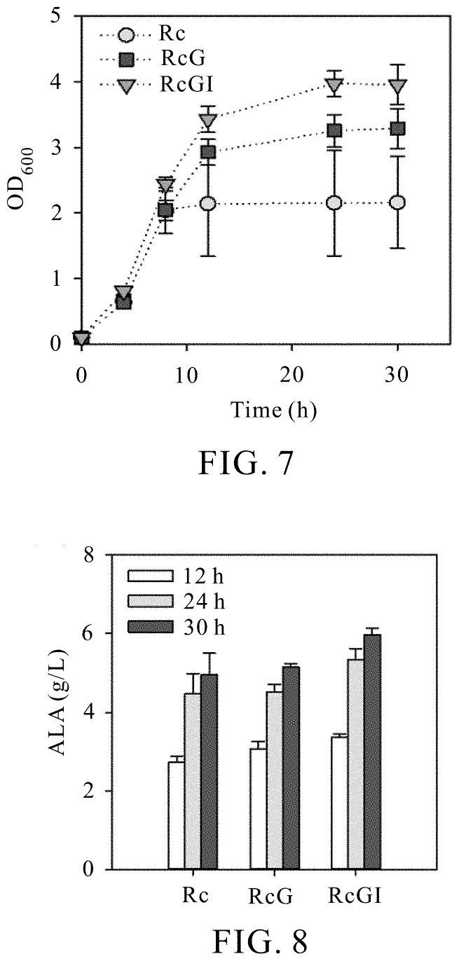

The growth rate of the RcGI strain and the ALA production efficiency test are shown in FIG. 7 and FIG. 8. The RcGI strain that directly integrates the RchemA gene, GroES gene and GroEL gene into the E. coli gene has a higher growth rate and ALA yield than other E. coli strains Rc and RcG that express the RchemA gene, GroES gene and GroEL gene via a plasmid.

Iron Ion Addition Test of RcGI Strain

First, the RcGI strain was prepared according to the aforementioned preparation method of the RcGI strain. The RcGI strain was inoculated into 2 mL of LB culture medium and cultured under 37° C. for 12 hours to serve as the pre-culture medium. The pre-culture medium of the RcGI strain was inoculated into four groups of 30 mL MM9 culture medium (containing 20 g/L glucose and 10 g/L glycerol as the carbon source, in which the weight ratio of glucose to glycerol is 2:1) in a 250 mL Erlenmeyer flask at an inoculation ratio of 2%. The above four groups of MM9 culture medium inoculated with the RcGI strain were named as the first group to the fourth group respectively. After the RcGI strains were inoculated respectively into the MM9 culture media of the first to the fourth groups, the first to the fourth groups were incubated under 37° C. with shaking at 200 rpm. When the concentration of the bacterial fluids of the first to the fourth groups were determined by spectrophotometer to reach an OD₆₀₀ value of approximately 0.6, 0.1 mM IPTG, 4 g/L glycine, 1 g/L succinic acid and 50 μM pyridoxal phosphate (PLP) were added respectively to the bacterial fluids of the first to the fourth groups. After adding IPTG, glycine, succinic acid and pyridoxal to the bacterial fluids of the first to the fourth groups, the culture of the first to the fourth groups was continued for 30 hours at a rotation speed of 300-400 rpm in an environment of 30° C., in which the culture medium of the first group was added with 0.4 mM ferric citrate at the beginning of culture, the culture medium of the second group was added with 0.4 mM ferric citrate after 3 hours of culture, the culture medium of the third group was added with 0.4 mM ferric citrate after 8 hours of culture, and the culture medium of the fourth group was added with 0.4 mM ferric citrate after 12 hours of culture. At the 4^{th}, 8^{th}, 12^{th}, 24^{th} and 30^{th} hour time points of culture, 200 μL of the bacterial fluids of the first to the fourth groups were taken to determine the OD₆₀₀ values thereof by a spectrophotometer. According to the ALA production efficiency test method of Example 1, the ALA yield of the RcGI strains of the first to the fourth groups were determined respectively at the 12^{th}, 24^{th} and 30^{th} hour after culture.

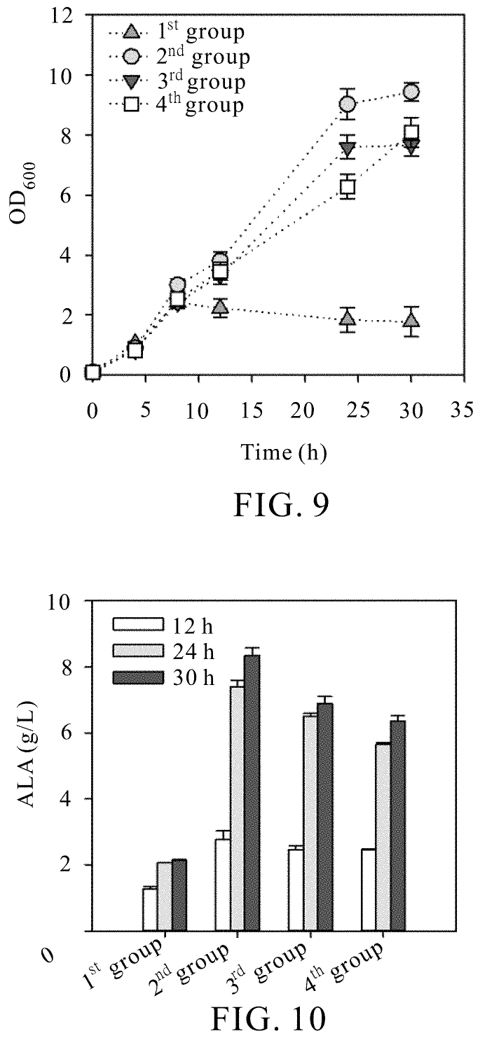

The test results are shown in FIG. 9 and FIG. 10. It can be seen that adding iron ions at the beginning of the culture will causes growth pressure on strains. When the time for adding iron ions is delayed, the growth rate of RcGI strains and ALA yield can be further improved.

Mass Production Efficiency Test of RcGI Strain in Fermentation Tank

First, the RcGI strain was prepared according to the aforementioned preparation method of the RcGI strain. Next, the RcGI strain was inoculated into 2 mL of LB culture medium and cultured under 37° C. for 12 hours to serve as the pre-culture medium. The pre-culture medium of the RcGI strain was inoculated into 300 mL of MM9 culture medium (containing 20 g/L glucose and 10 g/L glycerol as the carbon source, in which the weight ratio of glucose to glycerol is 2:1) in a 1 L small fermentation tank at the inoculation ratio of 2% for culture. The RcGI strains in the fermentation tanks were incubated under 37° C. with shaking at 200 rpm. When the concentration of the bacterial fluid in the fermentation tank reached an OD₆₀₀ value of approximately 0.6 determined by the spectrophotometer, 0.1 mM IPTG, 4 g/L glycine, 1 g/L succinic acid and 50 μM pyridoxal phosphate (PLP) were added respectively into the fermentation fluids in the fermentation tanks. After adding IPTG, glycine, succinic acid and pyridoxal into the fermentation tanks, the RcGI strains in the fermentation tanks continued the culture at a rotation speed of 300-400 rpm in an environment of 30° C. for 30 hours. Glycine was added into the fermentation fluids in the fermentation tanks after 12 hours of culture. At the beginning, the 4^{th}, 8^{th}, 12^{th}, 24^{th}, 26^{th}, 29^{th} and 30^{th} hour time points of culture, 200 μL of the bacterial fluids of the RcGI strains were taken to determine the OD₆₀₀ values thereof by the spectrophotometer. According to the ALA production efficiency test method of Example 1, the ALA yield of the RcGI strain was determined at each sampling time point. Also, the acetic acid content of the RcGI strain was determined at the aforementioned sampling time points. If no acetic acid is produced, it means that most of the carbon source enters the citric acid cycle pathway in the biological metabolism process, i.e., most of the carbon source is fully used in the production of ALA.

The mass production efficiency test results of the RcGI strain in the fermentation tank are shown in FIG. 11. In the mass production process in the fermentation tank, when the RcGI strain is cultured for the 30^{th} hour, the ALA yield can reach 15.6 g/L, and the production rate can reach 0.52 g/L/h.

E. coli A-RcYI of Example 3

Example 3 provides an E. coli A-RcYI. The A-RcYI strain contains RchemA gene, pdxY gene and additional inserted pRARE plasmids. The pRARE plasmid can provide tRNA to assist the performance of protein. Example 3 further tests the efficiency improvement of the production of ALA by E. coli via the synergistic effect of the RchemA gene and the pdxY gene with the increase of the expression of tRNA in E. coli. The preparation method of the A-RcYI strain of Example 3 is as follows.

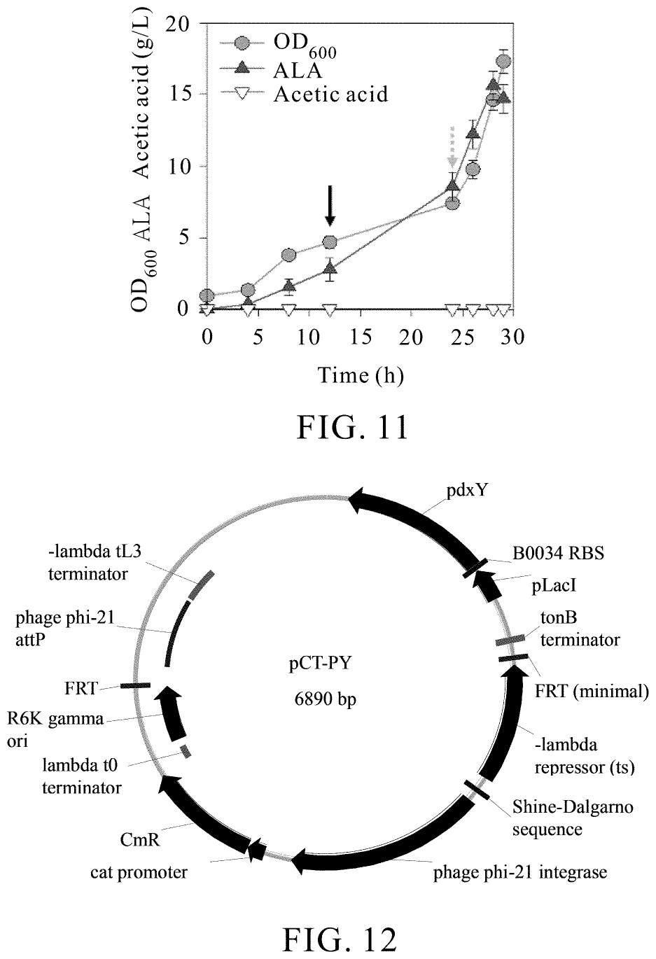

First, CRIM plasmids as shown in FIG. 1, pCT-PY plasmids as shown in FIG. 12 and pRARE plasmids and E. coli strain BL21 (DE3) were prepared. Next, the RchemA gene was inserted into the CRIM plasmid, and the pdxY gene entered into the E. coli via pCT-PY plasmid. According to the transformation method in Example 1, the CRIM plasmid with the RchemA gene was inserted into the HK022 site of E. coli BL21 (DE3), and the pCT-PY plasmid with the pdxY gene was inserted into the P21 site of E. coli BL21 (DE3). The pAH69 plasmid was then transferred into E. coli BL21 (DE3) according to the method of Example 1 to remove the kanamycin gene in the strain. Finally, according to the transformation method in Example 1, the pRARE plasmid was transferred into E. coli BL21 (DE3) to prepare the A-RcYI strain. At this time, the DNA of the A-RcYI strain had the sequences of SEQ ID NO. 1 and SEQ ID NO. 2 as shown in the sequence listing, and the A-RcYI strain also had pRARE plasmids inside the A-RcYI strain.

The above Example 3 reveals that the RchemA gene and the pdxY gene are transferred into the A-RcYI strain through the CRIM plasmid and the pCT-PY plasmid to express the RchemA gene and the pdxY gene, but in other examples, other known vectors, other promoters, or other methods can be used to make the E. coli strain express the RchemA gene and the pdxY gene, and is not limited to Example 3. In addition, the E. coli strain selected in Example 3 is BL21 (DE3), but in other embodiments, other E. coli strains can also be used to express RchemA gene, pdxY gene and pRARE plasmid.

Mass Production Efficiency Test of A-RcYI Strain in Fermentation Tank

First, the A-RcYI strain was prepared according to the aforementioned preparation method of the A-RcYI strain. Next, the A-RcYI strain was inoculated into 2 mL of LB culture medium and cultured under 37° C. for 12 hours to serve as the pre-culture medium. The pre-culture medium of the A-RcYI strain was inoculated into 300 mL of MM9 culture medium (containing 20 g/L glucose and 10 g/L glycerol as the carbon source, in which the weight ratio of glucose to glycerol is 2:1) in a 1 L small fermentation tank at the inoculation ratio of 2% for culture.

The A-RcYI strains in the fermentation tanks were incubated under 37° C. with shaking at 300-400 rpm. When the concentration of the bacterial fluid in the fermentation tank reached an OD₆₀₀ value of approximately 0.6 determined by the spectrophotometer, 0.1 mM IPTG, 6 g/L glycine, 1 g/L succinic acid and 50 μM pyridoxal were added respectively to the fermentation fluids in the fermentation tanks. After adding IPTG, glycine, succinic acid and pyridoxal into the fermentation tanks, the A-RcYI strains in the fermentation tanks continued the culture at a rotation speed of 300-400 rpm in an environment of 30° C. for 35 hours. Glycine with a final concentration of 6 g/L was added to the fermentation fluids in the fermentation tanks after 12 hours of culture. At the beginning, the 4^{th}, 8^{th}, 12^{th}, 24^{th}, 30^{th}, 32^{th} and 35^{th} hour time points of culture, the bacterial fluids of the A-RcYI strains were taken to determine the OD₆₀₀ values thereof by the spectrophotometer. According to the ALA production efficiency test method of Example 1, the ALA yield of the A-RcYI strain at each sampling time point was determined.

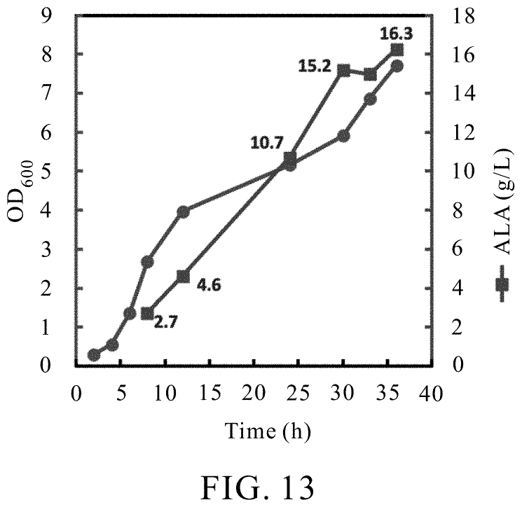

The mass production efficiency test results of the A-RcYI strain in the fermentation tank are shown in FIG. 13. In the mass production process in the fermentation tank, when the A-RcYI strain is cultured for the 35^{th} hour, the ALA yield can reach 16.3 g/L, and the production rate can reach 0.47 g/L/h.

As mentioned above, the above-mentioned E. coli capable of producing ALA through gene transfer includes PIECE strain, RcGI strain and A-RcYI strain, which are strains with fast growth rate and high ALA yield, and the method using these strains to produce ALA can produce ALA quickly with high yield.

The present invention has been disclosed in preferred embodiments above, but those skilled in the art should understand that these embodiments are only used to describe the present invention and should not be construed as limiting the scope of the present invention. It should be noted that all changes and substitutions equivalent to the embodiments should be included in the scope of the present invention. Therefore, the protection scope of the present invention should be defined by the claims.

What is claimed is:

1. An Escherichia coli having double pdxY gene and being capable of producing 5-aminolevulinic acid.

2. The Escherichia coli of claim 1, wherein the Escherichia coli further comprises RchemA gene.

3. The Escherichia coli of claim 2, wherein the Escherichia coli further comprises pRARE plasmid.

4. An Escherichia coli having the sequence of SEQ ID NO. 1 and being capable of producing 5-aminolevulinic acid.

5. The Escherichia coli of claim 4, wherein the Escherichia coli further has the sequence of SEQ ID NO. 2.

6. The Escherichia coli of claim 5, wherein the Escherichia coli further comprises pRARE plasmid.

7. A method for producing 5-aminolevulinic acid, comprising:
(a) providing the Escherichia coli having double pdxY gene or the sequence of SEQ ID NO. 1, where the Escherichia coli being capable of producing 5-aminolevulinic acid; and
(b) inoculating the Escherichia coli in a medium containing a carbon source, isopropyl-β-D-thiogalactoside, glycine, succinic acid and pyridoxal for culture to produce 5-aminolevulinic acid.

8. The method of claim 7, wherein the carbon source is a mixed carbon source composed of glucose and glycerol.

9. The method of claim 7, wherein the medium contains iron ions.
